(19) United States
(12) Patent Application Publication
(10) Pub. No.: US 2006/0035375 A1
(43) Pub. Date: Feb. 16, 2006

(54) Method for selectively culturing epithelial or carcinoma cells

(76) Inventors: Jonathan F. Head, Baton Rouge, LA (US); Robert L. Elliott, Baton Rouge, LA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/918,741

(22) Filed: Aug. 16, 2004

(51) Int. Cl. C12N 5/08; C12N 5/02

(52) U.S. Cl. 435/383; 435/366

(57) ABSTRACT

A method for selectively growing epithelial cells or carcinoma cells in vitro without fibroblast overgrowth comprises (a) suspending a cell pellet comprising digested epithelial or carcinoma cells in a first growth medium, the medium comprising D-valine MEM, methyl cellulose, fetal serum, glutamine and an antibiotic; wherein the methyl cellulose is present in the medium at a concentration sufficient to inhibit growth of fibroblast cells present in the cell pellet; (b) adding the suspension to a cell culture vessel comprising an inner surface which has been at least partially coated with an attachment medium comprising a protein extract, D-valine MEM, glutamine and an antibiotic; and (c) incubating the suspension in the coated vessel to allow selective growth of the epithelial cells or carcinoma cells.

### FIELD OF THE INVENTION

This invention relates to a method of culturing epithelial or carcinoma cells. More specifically, this invention relates to a method for selectively culturing and recovering such cells over fibroblast cells.

### BACKGROUND OF THE INVENTION

Many methods have been investigated to grow and isolate carcinoma cells and epithelial cells in cell culture such that pure cell lines can be obtained for use in chemosensitivity assays or vaccines. Although some success has been achieved with either growing or isolating the cells, it has proved difficult to develop a method that achieves both goals; typically either the desired cells could be purified from the culture medium but showed little growth or the desired cells multiplied but were overgrown by fibroblast cells. For example, growth of carcinoma cells in agar allows the formation of agar colonies without fibroblast overgrowth, but if the colonies are removed from the agar medium for further use they typically do not expand in culture (Fresney, R. I., Culture of Animal Cells: A Manual of Basic Techniques. Third Edition. Wiley-Liss, New York, 1994; p. 355). There also has been only limited success in propagating carcinoma cells purified in methyl cellulose.

Accordingly, it is a goal of this invention to provide a means of culturing epithelial or carcinoma cells without fibroblast cell overgrowth and then recovering the epithelial or carcinoma cells from the growth medium for further propagation.

### SUMMARY OF THE INVENTION

In accordance with this invention, a method for selectively growing epithelial cells or carcinoma cells in vitro comprises

(a) suspending a cell pellet comprising digested epithelial or carcinoma cells in a first growth medium, the medium comprising D-valine MEM, methyl cellulose, fetal serum, glutamine and an antibiotic; wherein the methyl cellulose is present in the medium at a concentration sufficient to inhibit growth of fibroblast cells present in the cell pellet;

(b) adding the suspension to a cell culture vessel comprising an inner surface which has been at least partially coated with an attachment medium comprising a protein extract, D-valine MEM, glutamine and an antibiotic; and

(c) incubating the suspension in the coated vessel to allow selective growth of the epithelial cells or carcinoma cells.

The cultured epithelial cells or carcinoma cells subsequently can be removed from the cell culture vessel with Trypsin-EDTA or other enzymes in a balanced salt solution.

### BRIEF DESCRIPTION OF THE FIGURES

FIGS. 1A and 1B show the growth of breast cancer cells from an enzyme digest of a breast tumor. FIG. 1A shows significant fibroblast contamination when the cells are grown in a conventional Eagle's Minimum Essential Medium with 10% fetal bovine serum; FIG. 1B shows fibroblast inhibition when the cells are grown in accordance with the method of the present invention.

FIGS. 2A and 2B show the growth of fibroblasts from an enzyme digest of human foreskin. FIG. 2A shows complete overgrowth of the surface of the medium by fibroblasts under normal, non-selective growth conditions (Eagle's Minimum essential Medium with 10% fetal bovine serum); FIG. 2B shows inhibition of fibroblasts when the cells are grown in accordance with the method of the present invention.

FIGS. 3A and 3B are photomicrographs of cells grown from a cell digest of a breast carcinoma. The cells in FIG. 3A were grown under normal conditions (Eagle's Minimum Essential Medium with 10% fetal bovine serum); those in FIG. 3B were grown using the method of the present invention. The cells in FIG. 3B show a lack of fibroblast growth around the carcinoma cell colonies.

FIGS. 4A and 4B are photomicrographs of cells grown from a cell digest of human foreskin. The cells in FIG. 4A were grown under normal conditions (Eagle's Minimum Essential Medium with 10% fetal bovine serum); those in FIG. 4B were grown in accordance with the method of the present invention. The cells in FIG. 4A show significant growth of fibroblasts; those in. FIG. 4B show an almost total lack of fibroblast growth.

### DETAILED DESCRIPTION OF THE INVENTION

The method of the present invention enables the selective growth of epithelial cells or carcinoma cells in vitro without concurrent fibroblast overgrowth. The method also provides for efficient recovery of the resultant epithelial or carcinoma cell colonies from the culture medium, such that the cells can be concentrated and used for further cell culturing or as a raw material for a subsequent process, such as the production of vaccines. Vaccine production using carcinoma cells to replace tumor-associated antigens is described, for example, in U.S. Pat. No. 5,478,556.

In accordance with the present invention, a sample of epithelial tissue or a tumor is obtained from a human or other mammal. The carcinoma sample can be obtained from any of a wide variety of cancers, including, for example, breast, prostate, pancreatic, lung, colo-rectal, gastric, testicular, ovarian, cervical, melanoma, retinoblastoma, or head and neck cancers. The tissue sample is digested in accordance with conventional procedures. Typically, small pieces of tissue in a sterile transport medium are digested with enzymes such as collagenase and Dnase.

A cell pellet of the resultant digest then is suspended in a first growth medium comprising D-valine MEM, supplemented with methyl cellulose, fetal calf serum, glutamine and an antibiotic. The methyl cellulose is present in the medium at a concentration sufficient to inhibit growth of fibroblast cells present in the cell pellet. Typically, the concentration of methyl cellulose is about 3.5 g. to about 10 g. per liter of D-valine MEM, preferably from about 6 g to about 8 g per liter of D-valine MEM and most preferably about 7 g per liter. D-valine MEM is available commercially, such as from Promocell (Heidelberg, Germany).

Typically, the D-valine MEM comprises from about 1% to about 15% of serum, about 0.25 mM to about 5 mM of glutamine and about 10 mg. to about 100 mg. of antibiotic per liter of MEM. Preferably, the medium comprises from about 5% to about 10% serum, from about 1 mM to about 3 mM of glutamine and about 20 mg to about 60 mg of antibiotic per liter of medium. Most preferably, the D-valine medium comprises about 10% serum, about 2 mM L-glutamine and about 50 mg. of antibiotic per liter of medium. A preferred serum is fetal bovine (calf) serum.

Suitable antibiotics include gentamicin, penicillin/streptomycin and any other antibiotic typically used in cell culture media. A preferred antibiotic is gentamicin.

The medium can comprise further additives and nutrients, such as a buffer/pH stabilizer and growth factors, such as epidermal growth factor, insulin, transferrin, hydrocortisone and/or estradiol. A buffer/pH stabilizer, such as HEPES, can be included in the medium in an amount sufficient to stabilize the pH without causing toxicity. Typically, the buffer/pH stabilizer is provided at a concentration of about 0.05 M to about 0.5 M, preferably at a concentration of about 0.1 M, per liter of medium. Insulin typically can be added at a concentration of about 1 mg to about 10 mg, preferably about 4 mg, per liter of medium. Epidermal growth factor typically can be added at a concentration of about 1 μg to about 10 μg, preferably about 5 μg, per liter. Transferrin typically can be provided at a concentration of about 5 mg to about 15 mg, preferably about 8 mg per liter. Hydrocortisone typically can be added at a concentration of about 1 μg to about 10 mg, preferably about 0.4 mg, per liter. Estradiol typically can be added at a concentration of about 0.1 mg to about 0.5 mg, preferably about 0.25 mg, per liter of medium.

The first growth medium comprising the cell pellet is vortexed, then added to a culture vessel, the inner surface of which has been at least partially coated with an attachment medium. This attachment medium comprises a protein extract diluted with D-valine MEM. Suitable protein extracts include Basement Membrane Extract, such as Cultrex Basement Membrane Extract, sold by R&D Systems, Minneapolis, Minn.), or other extracts that promote attachment of epithelial/carcinoma cells. Typically the volume:volume ratio of protein extract to D-valine MEM is within the range of about 2:1 to about 1:2, preferably about 1:1. The minimal essential medium is supplemented with conventional nutrients and additives, such as L-glutamine, growth factors and an antibiotic, but, contrary to the preceding step, is essentially free of serum. Essentially free of serum means that the medium contains less than about 0.75% serum, preferably less than 0.5% serum, and most preferably contains no serum.

The L-glutamine, growth factors and antibiotic can be added in the same relative amounts as set forth above in the discussion of the first growth medium. The medium further can comprise other additives, such as a buffer/pH stabilizer and one or more growth factors (transferrin, hydrocortisone, estradiol, epidermal growth factor, insulin), in the same relative amounts as set forth above in the discussion of the first growth medium.

The suspension of cells in methyl cellulose-containing first growth medium is incubated in the coated vessel for a period of time sufficient to allow selective growth of the epithelial cells or carcinoma cells. Typically this initial growth period is about 2-3 days in the culture vessel. The vessel is rinsed with a saline solution, such as Hanks Balanced Salt Solution, and the medium is replaced with a second growth medium comprising D-valine MEM. The minimal essential medium typically contains serum, L-glutamine and an antibiotic in the relative amounts set forth above in the discussion of the first growth medium. The medium further can contain additional additives and nutrients, such as those discussed above, in amounts comparable to those set forth above in the discussion of the first growth medium. Unlike the first growth medium, however, the second growth medium is at least essentially free of methyl cellulose. That is, the second growth medium contains less than about 3 g/L methyl cellulose, preferably less than about 1 g/L methyl cellulose, and most preferably contains no methyl cellulose.

The cells are incubated in the second growth medium for a time sufficient to allow further selective growth of the desired epithelial or carcinoma cells. Preferably, the cells are incubated until the cell colonies reach a size of about 5 mm in diameter or fibroblasts begin to fill in the spaces between the colonies.

Once the cells have been grown to the desired point, the growth medium is removed and the cells are washed. Fibroblast cells present can be removed from the attached surface, leaving behind the attachment medium and the desired epithelial cells or carcinoma cells, through the addition of an isotonic buffer salt solution, such as HBSS without calcium or magnesium, and/or Trypsin-EDTA.

Once fibroblast cells, if any are present, have been selectively removed, the desired epithelial cells or carcinoma cells can be removed from the attached surface. A preferred method of recovering the cells is with an enzyme, such as pronase or dispase. The cells then can be replated in a conventional medium and cultured in accordance with conventional procedures to create transferable epithelial/carcinoma monolayer cultures for subsequent use in the preparation of vaccines, chemosensitivity/chemoresistance assays, etc.

It has been found that by using the culturing method of the present invention, the growth of fibroblasts is inhibited by at least 30%, preferably at least 75%, and more preferably by at least 95% in comparison to conventional culturing methods.

The invention is described further in the following examples, which are provided for illustrative purposes only and are not intended to be construed as limiting.

### EXAMPLES

### Example 1

### Preparation of Media Additives

Preparation of Working Solution of Epidermal Growth Factor and Insulin

Materials

Insulin—Product # 2767 from Sigma (St. Louis, Mo.), 100 mg/vial. Store at −20° C. upon receipt.

Epidermal Growth Factor (EGF)—Product # E9964 from Sigma (St. Louis, Mo.), 200 micrograms. Store at −20° C. upon receipt.

12×75 sterile snap cap tubes, Falcon # 352032 from Fisher Scientific (Houston, Tex.).

Sterile distilled water.

Procedure

Add 50 ml sterile distilled water to vial of insulin.

Add 20 ml sterile distilled water to vial of EGF.

Aliquot 1.0 ml of insulin and 0.25 ml of EGF into each 12×75 snap cap tube.

Store at −70° C.

Expiration date: 3 months after reconstitution.

Preparation of Working Dilution of Transferrin Materials

Transferrin—Product # T 0665, Sigma (St. Louis, Mo.), 1 gram; store at 2-8° C. upon receipt.

1× Hank's Balanced Salt Solution (HBSS), Product # H 8264, Sigma (St. Louis, Mo.), 500 ml.

0.22 micron filter—Product # 09-719A, Fisher Scientific (Houston, Tex.).

12×75 sterile snap cap tubes, Falcon # 35032, Fisher Scientific (Houston, Tex.).

Procedure

Add 100 ml of 1×HBSS to 200 mg of transferrin.

Filter sterilize through 0.22 micron filter.

Aliquot 2 ml into each 12×75 mm sterile tube.

Freeze at −70° C.

Expiration date: 3 months after reconstitution.

Preparation of Working Dilution of Hydrocortisone and Estradiol

Materials

Hydrocortisone—Product # H 0888, Sigma (St. Louis, Mo.), 1 gram; store at room temperature upon receipt.

Estradiol—Product # E 2758, Sigma (St. Louis, Mo.), 1 gram; store at room temperature upon receipt.

95% Ethanol

12×75 sterile snap cap tubes, Falcon # 34032, Fisher Scientific (Houston, Tex.).

Procedure

Add 30 ml 95% ethanol to 30 mg hydrocortisone.

Add 50 ml 95% ethanol to 6.8 mg of estradiol.

Aliquot 0.20 ml. of hydrocortisone and 1.00 ml of estradiol into each 12×75 mm test tube.

Store at −20° C.

Expiration date: 3 months after reconstitution.

### Example 2

### Preparation of Attachment Medium

Materials and Content

D-valine MEM—PromoCell (PromoCell; Heidelberg, Germany), 500 ml; store at 4° C.

L-glutamine (200 mM)—Product # G 7513, Sigma (St. Louis, Mo.), 5 ml, store at −20° C. upon receipt

HEPES (1M)—Product # H 0887, Sigma, 5 ml; store at 2-8° C. upon receipt

Insulin and EGF, 1.25 ml (see above)

Transferrin, 2.0 ml (see above)

Hydrocortisone and estradiol, 1.20 ml (see above)

Gentamicin (10 mg/ml)—Product #G 1272, sigma, 2.5 ml; store at 2-8° C. upon receipt.

Cultrex Basement Membrane Extract—R&D Systems (Minneapolis, Minn.); store at −20° C. upon receipt.

Procedure

To 500 ml of D-valine MEM add 5 ml of L-glutamine (200 mM), 5 ml of HEPES (1M), 1.25 ml of insulin and EGF, 2.0 ml of transferrin, 1.20 ml of hydrocortisone and estradiol, 2.5 ml of gentamicin (10 mg/ml); store at 2-8° C.

Expiration date: 3 months after reconstitution.

To 2.5 ml of cultrex Basement Extract on ice add 2.5 ml of above medium at 2-8° C. Use immediately.

### Example 3

### Preparation of First Growth Medium

Materials

D-valine MEM—PromoCell (PromoCell; Heidelberg, Germany), 500 ml; store at 4° C.

Fetal bovine serum—Product # F6178, Sigma, 50 ml; store at −20° C. upon receipt

L-glutamine (200 mM)—Product # G 7513, Sigma (St. Louis, Mo.), 5 ml, store at −20° C. upon receipt

HEPES (1M)—Product # H 0887, Sigma, 5 ml; store at 2-8° C. upon receipt

Insulin and EGF, 1.25 ml (see above)

Transferrin, 2.0 ml (see above)

Hydrocortisone and estradiol, 1.20 ml (see above)

Gentamicin (10 mg/ml)—Product #G 1272, Sigma, 2.5 ml; store at 2-8° C. upon receipt

Methyl cellulose—Product #M 0512, Sigma (St. Louis, Mo.), 100 g. Store at room temperature upon receipt.

Procedure

Complete D-valine MEM:

To 500 ml of D-valine MEM add 50 ml of fetal bovine serum, 5 ml of L-glutamine (200 mM), 5 ml of HEPES (1M), 1.25 ml of insulin and EGF, 2.0 ml of transferrin, 1.20 ml of hydrocortisone and estradiol, 2.5 ml of gentamicin (10 mg/ml). Store at 2-8° C. Expiration date: 3 months after reconstitution.

Sterilize 3.5 g methyl cellulose in a 500 ml glass medium bottle with a Teflon®-coated stirring bar inside. Add 150 ml of complete D-valine MEM. Stir on a magnetic stirrer until the methyl cellulose is dissolved. Add an additional 350 ml of complete D-valine MEM. Stir until uniform consistency. Store at 2-8° C. Expiration date: 3 months after reconstitution.

### Example 4

### Preparation of Second Growth Medium

The same procedures are followed as for the preparation of complete D-valine MEM in Example 3 except that the second growth medium does not include methyl cellulose.

Materials

D-valine MEM—PromoCell (PromoCell; Heidelberg, Germany), 500 ml; store at 4° C.

Fetal bovine serum—Product # F6178, Sigma, 50 ml; store at −20° C. upon receipt

L-glutamine (200 mM)—Product # G 7513, Sigma (St. Louis, Mo.), 5 ml, store at −20° C. upon receipt

HEPES (1M)—Product # H 0887, Sigma, 5 ml; store at 2-8° C. upon receipt

Insulin and EGF, 1.25 ml (see above)

Transferrin, 2.0 ml (see above)

Hydrocortisone and estradiol, 1.20 ml (see above)

Gentamicin (10 mg/ml)—Product #G 1272, Sigma, 2.5 ml; store at 2-8° C. upon receipt

Procedure

Complete D-valine MEM:

To 500 ml of D-valine MEM add 50 ml of fetal bovine serum, 5 ml of L-glutamine (200 mM), 5 ml of HEPES (1M), 1.25 ml of insulin and EGF, 2.0 ml of transferrin, 1.20 ml of hydrocortisone and estradiol, 2.5 ml of gentamicin (10 mg/ml). Store at 2-8° C. Expiration date: 3 months after reconstitution.

### Example 5

### Preparation of a Cell Pellet

A piece of breast tumor tissue (1 cubic centimeter or approximately 1 gram) obtained from a female breast cancer patient at biopsy was cut into 3 pieces. The pieces were chilled in 15 ml of sterile transport medium (alpha-MEM, 10% fetal calf serum, 2 mM L-glutamine, 50 mg/L gentamicin) at 4° C. Upon arrival at the laboratory, the chilled tissue and transport medium was poured into a petri dish and the tissue cut into small pieces (about 1 mm cubes) in the chilled transport medium. The chilled medium containing the chilled small pieces of breast tumor tissue was poured into a spinner flask. 4.5 ml of 3% collagenase (Collagenase, type 3, Product # CLS-3, Worthington Biochemical, Lakewood, N.J.) and 4.5 ml of 0.02% Dnase (Deoxyribonuclease I, Type IV, Product #D 5025, Sigma, St. Louis, Mo.) were added to the medium. The resulting enzyme digest was incubated with spinning for 5 hours at 37° C. The incubated digest was poured into a 50 ml conical centrifuge tube and brought to 40 ml with Hanks Balanced Salt Solution (HBSS) without calcium or magnesium (Product # H 6648, Sigma, St. Louis, Mo.). The number of the cells in the 40 ml cell suspension was counted with hemocytometer (seeking about 250,000 cells to initiate the culture). The 40 ml cell suspension was centrifuged at 1000 g for 15 minutes. The resulting supernatant was discarded and the pellet of breast tumor cells was collected.

### Example 6

### Selective Growth Of Carcinoma Cells

The pellet of cells was resuspended in 10 ml of the first growth medium as described in Example 3 (7 g/liter methyl cellulose in D-valine MEM, 10% fetal calf serum, 2 mM L-glutamine, 50 mg/L gentamicin, 10 mM HEPES, 4.0 mg/L insulin, 5.0 μg/L epidermal growth factor (EGF), 8.0 mg/L transferrin, 0.4 mg/L hydrocortisone, 0.27 mg/L estradiol).

A T-75 culture flask was coated with 5.0 ml of a solution containing 2.5 ml of the attachment medium described in Example 2 above (2.5 ml of Cultrex Basement Membrane Extract (R&D Systems; Minneapolis, Minn.) diluted with 2.5 ml of a D-valine MEM solution (D-valine MEM, 2 mM L-glutamine, 50 mg/L gentamicin, 10 mM HEPES, 4.0 mg/L insulin, 5.0 μg/L epidermal growth factor (EGF), 8.0 mg/L transferrin, 0.4 mg/L hydrocortisone, 0.27 mg/L estradiol).

The cells suspended in the first growth medium were incubated in the coated T-75 flask for 3 days. After incubation, the growth medium was discarded, and the incubated cells were rinsed with 10 ml HBSS.

The rinsed cells were incubated in 10 ml of the second growth medium, prepared as described in Example 4, above, until the size of colonies became about 5 mm in diameter, which is about the size of a pencil eraser, or fibroblasts began to fill in the spaces between colonies, whichever was earlier. The second growth medium was discarded after the second incubation, and the cells were rinsed twice with 10 ml of HBSS without calcium or magnesium. When necessary, contaminating fibroblasts in the incubated cell culture were removed by treating the cell culture with 10 ml of a trypsin-EDTA solution (2.5 g. porcine trypsin and 0.2 g EDTA/L Sigma #T 4049, St. Louis, Mo.). The trypsin-EDTA treated cells were washed with 10 ml of HBSS without calcium or magnesium to wash off the remaining fibroblasts from the trypsin-EDTA treatment. The trypsin-EDTA treated carcinoma cells were recovered by adding dispase (2 units/ml in 10% FBS, α-MEM for up to 60 minutes). The cells were spun down at 1000 g for 15 minutes and replated in 10 ml of 10% FBS, α-MEM in a T-75 flask to establish the cells in continuous cell culture.

### Example 7

### Results of Comparative Tests

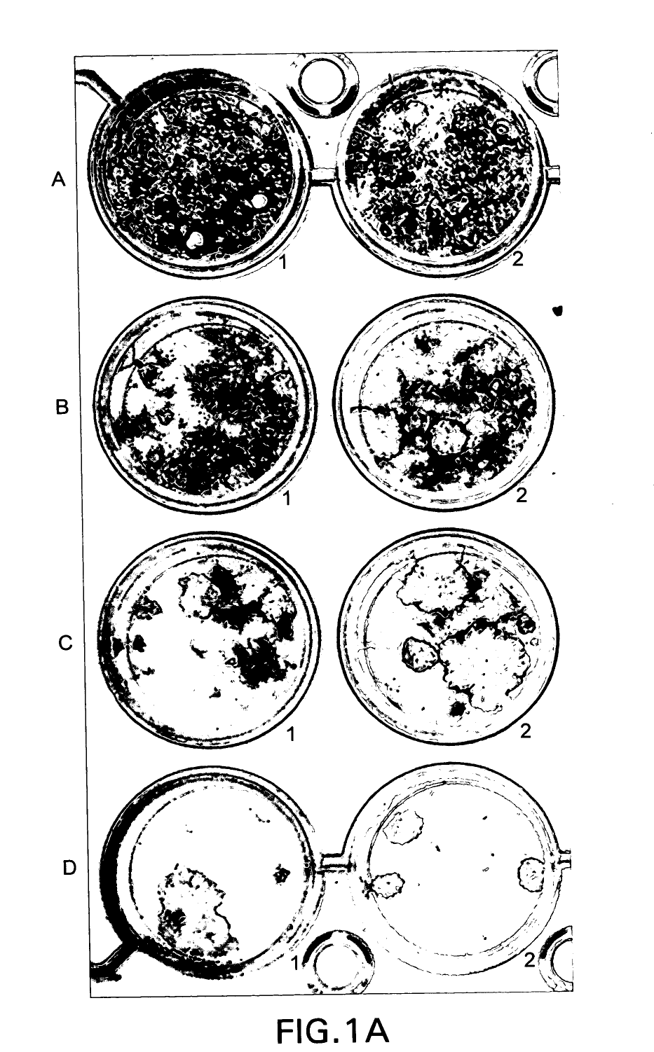

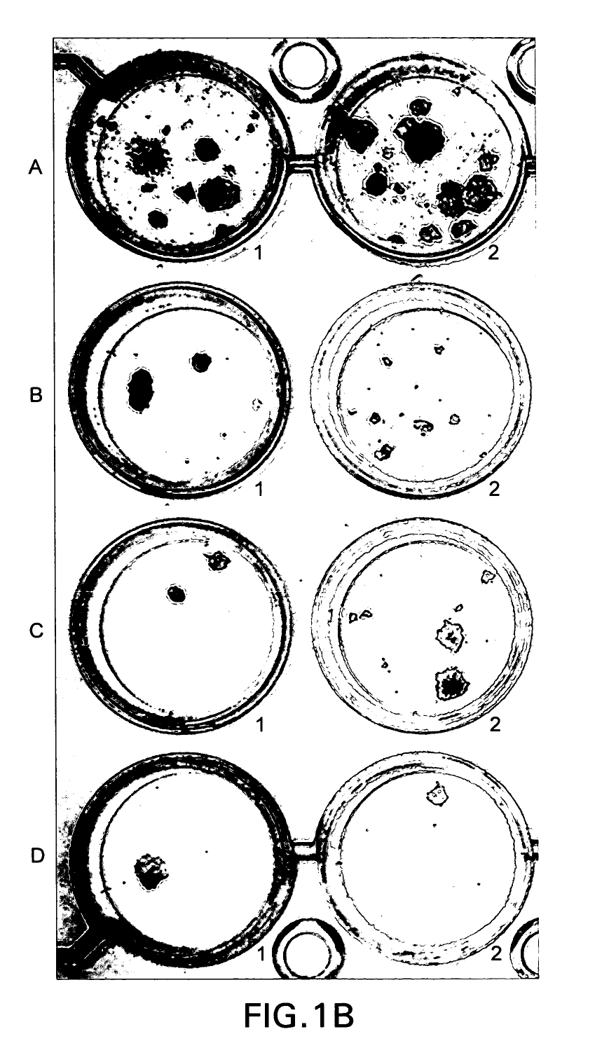

The advantages of growing cells according to the method of this invention are illustrated in the Figures provided herewith. FIGS. 1A and 1B show the gross fibroblast contamination for breast tumor cells prepared as in Example 5 and then grown in either a conventional medium (control) or as in Example 6, respectively. The control sample was grown in conventional α-MEM, rather than the D-val MEM of the present invention. Only the wells used for the selective growth method were coated with 150 μl of the attachment medium described above.

In each instance the carcinoma cells were suspended in the growth medium at a concentration of 25,000 cells/ml. One ml was pipetted into the first two wells of the first row, 0.5 ml into the first two wells of the second row, 0.25 ml into the first two wells of the third row and 0.125 ml into the first two wells of the fourth row. The total volume of all wells was brought to 1.0 ml with growth medium. The cells were incubated for three days and then washed 2 times with 1.0 ml HBSS. One ml of complete D-valine MEM was added to each well of the plate of FIG. 1A and one ml of α-MEM to each well of the plate of FIG. 1B. After 7 days in culture, the cells in each plate again were washed with 1.0 ml of HBSS, fixed in 70% ethanol for 20 minutes and then air dried overnight. The fixed plates were stained with crystal violet by submerging them in a crystal violet solution prepared by dissolving 1 g of crystal violet in 400 ml of 95% ethanol and then adding 1600 ml tap water for two minutes. The plates were rinsed in running tap water and allowed to air dry.

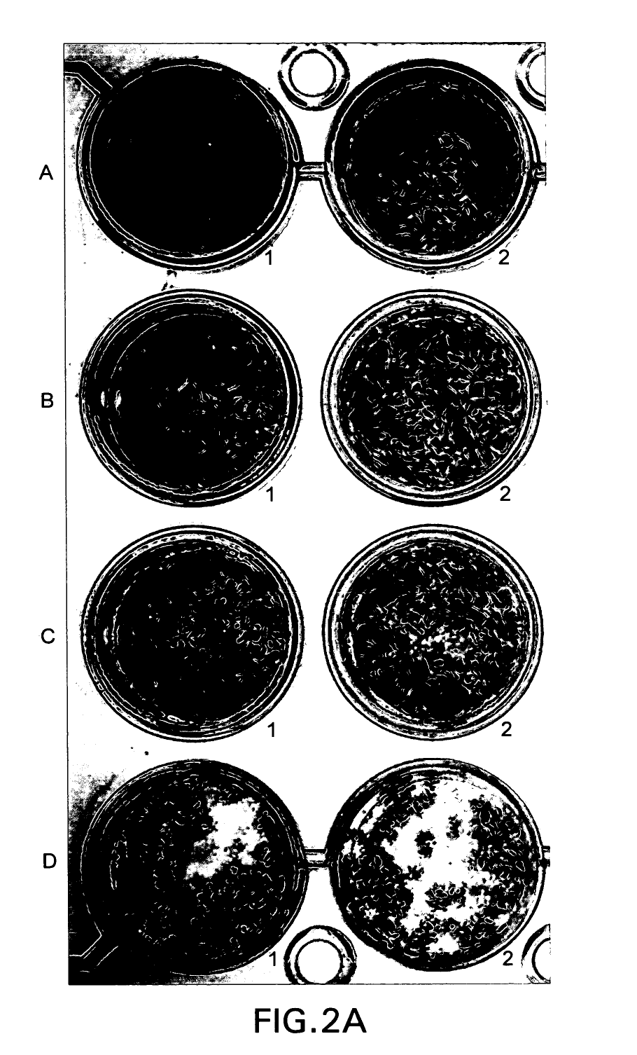

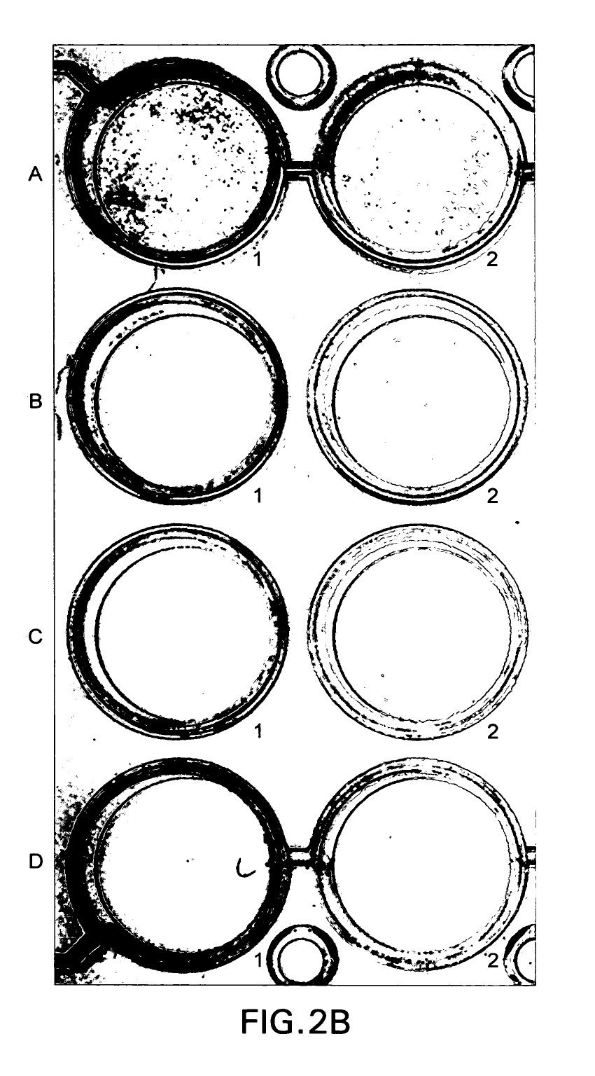

In a second comparative experiment, an enzyme digest of human foreskin was cultured in accordance with the present invention and in α-MEM. FIGS. 2A and 2B show the fibroblast growth which occurred in the wells of a plate in which cells were grown using conventional non-selective conditions and using the selective conditions of the present invention, respectively. As shown in the Figure, there was complete overgrowth of the well surface by fibroblasts under the conventional nonselective conditions, but significant inhibition of fibroblasts using the selective method of the invention. The human foreskin was obtained from a human infant's circumcision and was digested by the same method as in the preceding example. The culture methods were the same as in the comparative experiment of FIG. 1. Plates were fixed and stained as above.

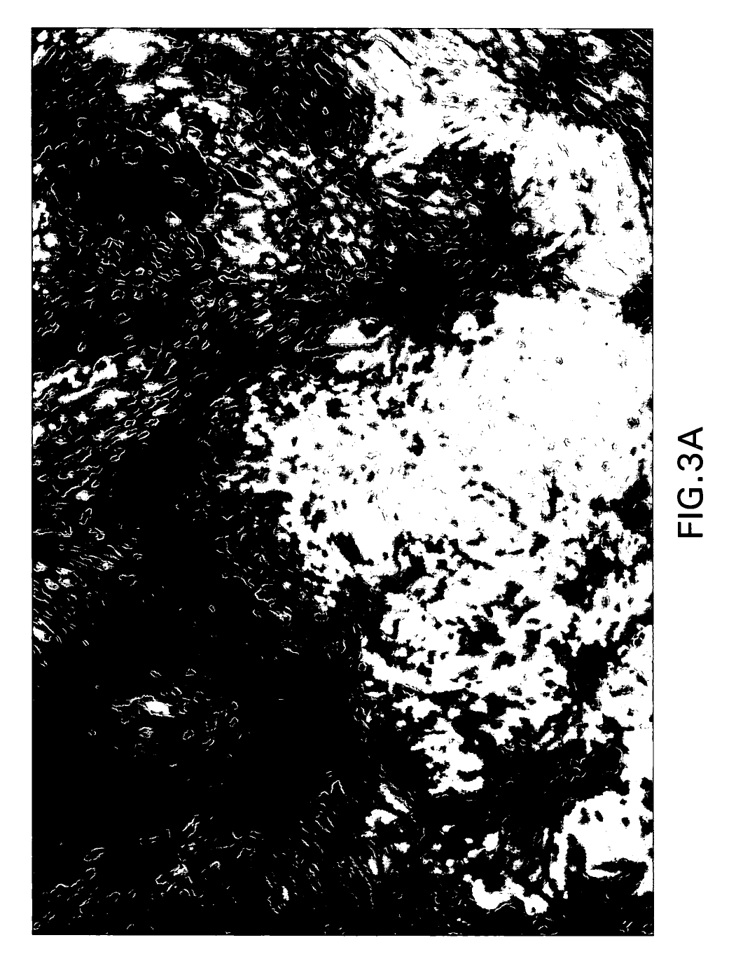

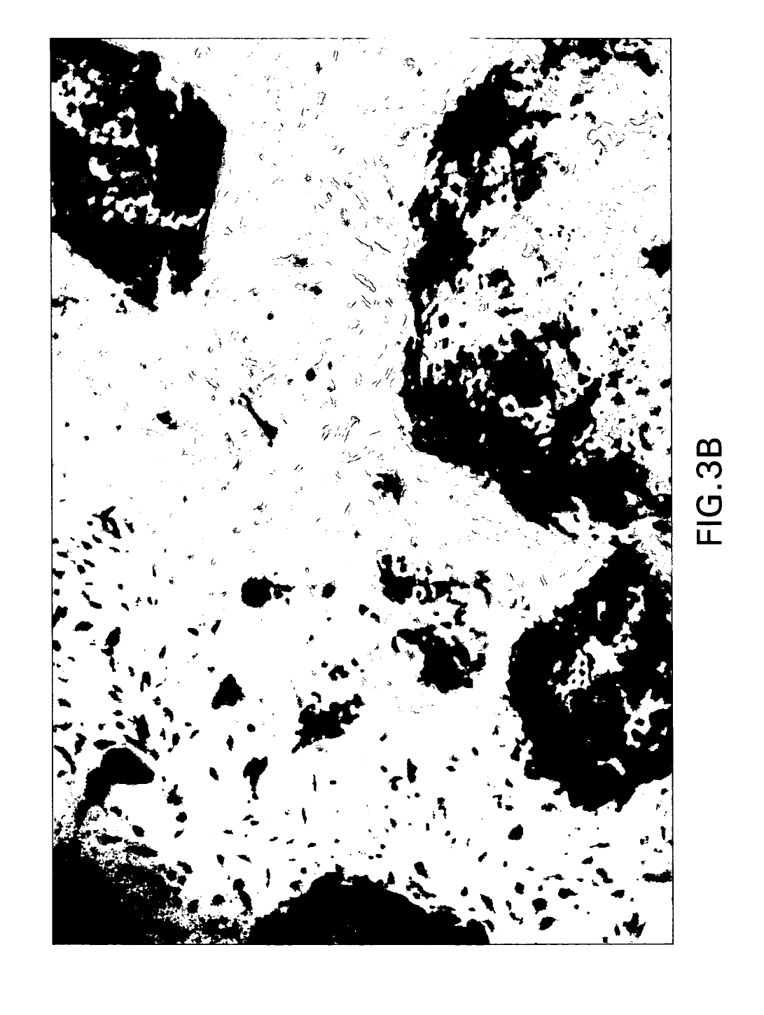

A third comparative experiment again compared the growth of human breast cancer cells under conventional conditions and in the selective medium of the present invention. Photomicrographs were made of the cells cultured in the first comparative example above. FIG. 3A and 3B are photomicrographs of the cells grown under conventional conditions and the selective conditions of the invention, respectively. FIG. 3B shows a lack of fibroblast growth around the colonies of tumor cells in comparison to that observed in FIG. 3A. The cells in the wells of the plates were fixed and stained as above and observed with an inverted microscope at 120×.

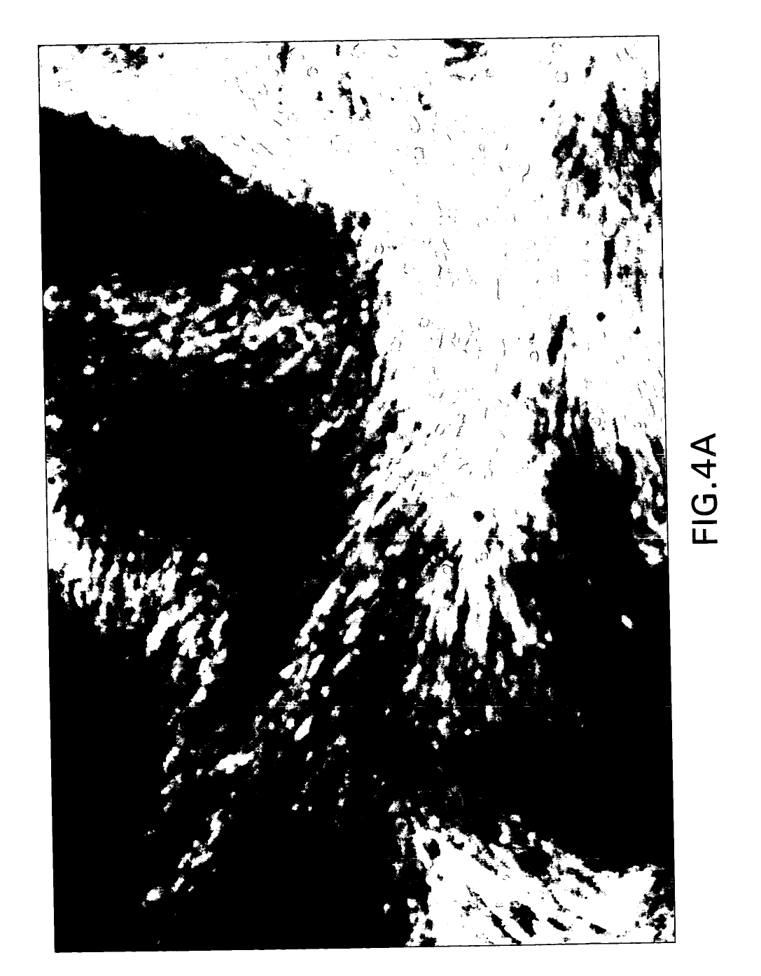

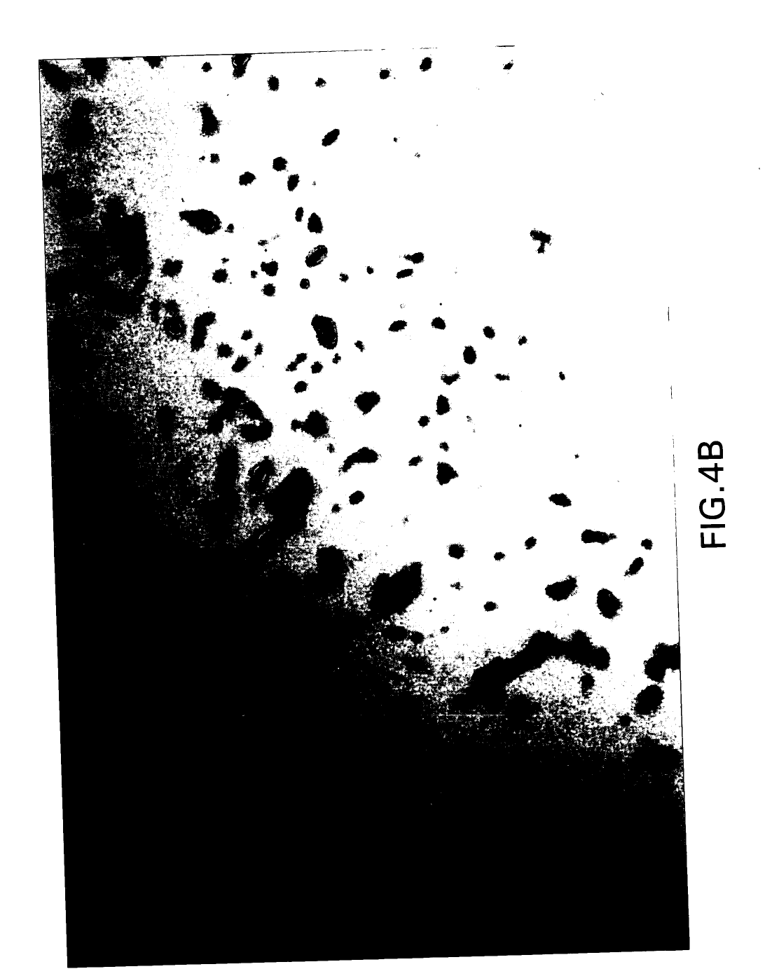

A fourth comparative experiment again compared the growth of cells of human foreskin under conventional conditions and under the selective conditions of the present invention. Photomicrographs were made of the cells cultured in the second comparative example above. FIGS. 4A and 4B are photomicrographs of the cells grown under conventional conditions and the selective conditions of the invention, respectively. FIG. 4A shows tremendous growth of swirling fibroblasts; FIG. 4B shows an almost total lack of fibroblast growth. The cells in the wells of the plates were fixed and stained as above and observed with an inverted microscope at 120×.

We claim:

1. A method for selectively growing epithelial cells or carcinoma cells in vitro comprising
(a) suspending a cell pellet comprising digested epithelial or carcinoma cells in a first growth medium, said medium comprising D-valine MEM, methyl cellulose, serum, glutamine and an antibiotic; wherein said methyl cellulose is present in said medium at a concentration sufficient to inhibit growth of fibroblast cells present in said cell pellet;
(b) adding said suspension to a cell culture vessel comprising an inner surface which has been at least partially coated with a medium comprising a protein extract, D-valine MEM, glutamine and an antibiotic; and
(c) incubating said suspension in said coated vessel to allow selective growth of said epithelial cells or carcinoma cells.

2. The method of claim 1, wherein said methyl cellulose is present in said first growth medium at a concentration in the range of about 3.5 to about 10 g/liter of D-valine MEM.

3. The method of claim 1, which further comprises rinsing said epithelial or carcinoma cells with a balanced salt solution, replacing said first growth medium with a second growth medium, wherein second growth medium comprises D-valine MEM, serum, glutamine and an antibiotic but is essentially free of methyl cellulose.

4. The method of claim 1, wherein said second growth medium is free of methyl cellulose.

5. The method of claim 1 or 3, wherein Trypsin-EDTA is added to said cell culture vessel to remove fibroblast cells which grow with said epithelial cells or carcinoma cells in said cell culture vessel.

6. The method of claim 1 or 5, wherein epithelial cells or carcinoma cells grown in said vessel are recovered from said growth medium with an enzyme.

7. The method of claim 6, wherein said enzyme is a pronase or dispase or Trypsin-EDTA.

8. The method of claim 1, wherein said first growth medium further comprises a buffer or pH stabilizer.

9. The method of claim 8, wherein said buffer or pH stabilizer is HEPES.

10. The method of claim 1, wherein said first growth medium further comprises a growth factor.

11. The method of claim 10, wherein said growth factor comprises insulin, epidermal growth factor, transferrin, hydrocortisone, estradiol or a combination thereof.

12. A cell culture composition comprising D-valine MEM and methyl cellulose;
wherein per liter of D-valine MEM, said composition comprises sufficient methyl cellulose to inhibit fibroblast growth when said composition is used as a growth medium for a cell sample containing fibroblasts.

13. The cell culture composition of claim 12, which further comprises serum, glutamine and an antibiotic;
wherein per liter of D-valine MEM said composition comprises from about 1% to about 15% serum, from about 0.25 mM to about 5 mM glutamine, and from about 10 mg/L to about 100 mg/L of an antibiotic.

14. The cell culture composition of claim 13, wherein said serum is fetal bovine serum.

15. The cell culture composition of claim 12 or 13, which further comprises a buffer or pH stabilizer.

16. The cell culture of claim 15, wherein said buffer or pH stabilizer is HEPES.

17. The cell culture composition of claim 12 or 13, which further comprises a growth factor.

18. The cell culture composition of claim 17, wherein said growth factor comprises insulin, epidermal growth factor, transferrin, hydrocortisone, estradiol or a combination thereof.

19. The cell culture composition of claim 18, wherein said insulin is present from about 1 mg/L to about 10 mg/L, said epidermal growth factor is present from about 1 μg/L to about 10 μg/L, said transferrin is present from about 5 mg/L to about 15 mg/L, said hydrocortisone is present from about 1 μg/L to about 10 mg/L, said estradiol is present from about 0.1 mg/L to about 0.5 mg/L.

20. A cell culture composition comprising D-valine MEM, methyl cellulose, serum, glutamine, an antibiotic, a buffer or pH stabilizer, and a growth factor; said growth factor comprising insulin, epidermal growth factor, transferrin, hydrocortisone, estradiol or a combination thereof;
wherein per liter of D-valine MEM, said composition comprises from about 3.5 g/l to about 10 g/l methyl cellulose, from about 1% to about 15% serum, from about 0.25 mM to about 5 mM glutamine, from about 10 mg/L to about 100 mg/L of an antibiotic; from about 0.05 M to about 0.5 M buffer or pH stabilizer, from about 1 mg/L to about 10 mg/L insulin, from about 1 μg/L to about 10 μg/L epidermal growth factor, from about 5 mg/L to about 15 mg/L transferrin, from about 1 μg/L to about 10 mg/L hydrocortisone, from about 0.1 mg/L to about 0.5 mg/L estradiol.

21. A cell culture composition comprising a protein extract and D-valine MEM;
wherein the volume:volume ratio of protein extract to D-valine MEM is within the range of about 2:1 to about 1:2.

22. The cell culture composition of claim 21, which further comprises L-glutamine, an antibiotic and a growth factor; wherein said composition is essentially free of serum.

23. The cell culture composition of claim 22, wherein said composition is free of serum.

24. The cell culture composition of claim 22, said composition comprising from about 0.25 mM to about 5 mM L-glutamine and from about 10 mg/L to about 100 mg/L of said antibiotic.

25. The cell culture composition of claim 22, wherein said growth factor comprises insulin, epidermal growth factor, transferrin, hydrocortisone, estradiol, or a combination thereof.

26. The cell culture composition of claim 25, wherein said insulin is present at a concentration of from about 1 mg/L to about 10 mg/L, said epidermal growth factor is present at a concentration of from about 1 μg/L to about 10 μg/L, said transferrin is present at a concentration of from about 5 mg/L to about 15 mg/L, said hydrocortisone is present at a concentration of from about 1 μg/L to about 10 mg/L, said estradiol is present at a concentration of from about 0.1 mg/L to about 0.5 mg/L.

27. A method for inhibiting growth of fibroblasts in a cell culture comprising epithelial or carcinoma cells which comprises
(a) obtaining a cell pellet comprising digested epithelial cells or carcinoma cells, said pellet further comprising fibroblast cells;
(b) suspending said cell pellet in a first growth medium, said medium comprising D-valine MEM, methyl cellulose, serum, glutamine and an antibiotic;
(c) adding said suspension to a cell culture vessel comprising an inner surface which has been at least partially coated with a medium comprising a protein extract, D-valine MEM, glutamine and an antibiotic; and
(d) incubating said suspension in said coated vessel such that said epithelial cells or carcinoma cells grow and fibroblast cell growth is inhibited.

28. The method of claim 27, wherein fibroblast cell growth is inhibited by at least 30%.

29. The method of claim 28, wherein fibroblast cell growth is inhibited by at least 75%.

30. The method of claim 29, wherein fibroblast cell growth is inhibited by at least 95%.
